(19) United States
(12) Patent Application Publication
(10) Pub. No.: US 2018/0125916 A1
(43) Pub. Date: May 10, 2018

(54) System and Method for Natural Hair Loss Prevention Oil

(71) Applicant: Zevi Schwab, Brooklyn, NY (US)

(72) Inventor: Zevi Schwab, Brooklyn, NY (US)

(21) Appl. No.: 15/343,213

(22) Filed: Nov. 4, 2016

(51) Int. Cl. A61K 36/87; A61K 36/15; A61K 36/53; A61K 36/534; A61K 36/185

(52) U.S. Cl. CPC A61K 36/87; A61K 36/15; A61K 36/185; A61K 36/534; A61K 36/53

(57) ABSTRACT

A first composition of essential oils, including 0.3% each of cedarwood, lavender, rosemary, thyme, and peppermint essential oils are thoroughly combined until evenly distributed. A second composition of oils are thoroughly combined, including 9.85% jojoba oil, 9.85% argan oil, and 78.79% grapeseed oil until evenly distributed. The first composition of essential oils is gradually stirred into the second composition of oils, producing a combined oil composition. The scalp is coated with the resulting combined oil composition, followed by massaging the oil composition into the scalp with kneading, squeezing and pinching motions, for 1-3 minutes and subsequently maintaining the oil mixture on the scalp for at least 30 minutes.

### FIELD OF THE DISCLOSED TECHNOLOGY

The disclosed technology relates generally to the treatment and/or prevention of hair loss (specifically, male pattern baldness).

### BACKGROUND OF THE DISCLOSED TECHNOLOGY

A normal hair growth cycle consists of three phases including the Anagen (growth phase), Catagen (transitional phase), and Telogen (resting phase). At any given time in healthy hair follicles and scalp, most hair should be growing and in the Anagen phase. Each hair spends several years in the Anagen phase. In the Catagen phase, the hair enters a transitional phase following the Anagen phase where hair growth slows and the hair follicle shrinks. Once the hair enters the Telogen phase, hair growth stops and the old hair detaches from the hair follicle. A new hair begins the growth phase, pushing the old hair out.

By age 50, approximately 85% of men and 40-50% of women suffer from significant hair loss. A variety of sources may cause hair follicles in men and women to shrink, destruct, and/or arrest normal scalp and hair follicle activity. Hair follicles become clogged with foreign bodies and/or pus due to infection. The overproduction of dihydrotestosterone (DHT) in the scalp of males causes hair follicles to shrink and die. The overproduction of sebum on the scalp causes clogged hair follicles that are unable to continue in their normal growth phase. Inadequate blood supply and improper nutrient intake and/or delivery to hair follicles and scalp result in hair loss.

Currently, the most common ways to treat male pattern baldness are through medication. There are two well-known treatments: Minoxidil and Finasteride, known under their trademark names Rogaine® and Propecia®, respectively. Minoxidil can take 6 months to begin working, and can cause shedding in the first 2 months of use. While the mechanism through which minoxidil supports hair growth is not fully understood, hypothetically, by expanding blood vessels and opening potassium channels, more oxygen, blood, and nutrients are able to reach the follicle. Minoxidil is less effective when there is a large area of hair loss. Minoxidil use is indicated for central (vertex) hair loss only and does not affect receding hairlines Minoxidil has a high likelihood of a considerable itching as a side effect. Other side effects of Minoxidil include unwanted hair growth elsewhere on the body, exacerbation of hair loss, sudden and unexplained weight gain, or swelling of the hands and feet.

Finasteride is known to cause permanent sexual dysfunction, possibly due to the fact that Finasteride is administered orally and thus affects DHT levels throughout the body, and not just in the scalp. Gynecomastia and depression have also been documented side effects of Finasteride. Clearly, there exists a need for a natural hair loss treatment without harmful side effects, with no obvious solution presently known in the prior art.

### SUMMARY OF THE DISCLOSED TECHNOLOGY

In one embodiment of the disclosed technology, a composition for treatment and/or prevention of hair loss includes cedarwood essential oil, lavender essential oil, rosemary essential oil, thyme essential oil, peppermint essential oil, jojoba oil, argan oil, and grapeseed oil.

In one embodiment, a composition for treatment and prevention of hair loss includes a mixture of essential oils including 0.3-5% cedarwood essential oil, 0.3-5% lavender essential oil, 0.3-5% rosemary essential oil, 0.3-5% thyme essential oil, and 0.3-5% peppermint essential oil, and a base oil mixture of 9.85-16.85% jojoba oil, 9.85-16.85% argan oil, and 68.79-88.79% grapeseed oil.

In further embodiments, the essential oil mixture is mixed independently until the oils in the essential oil mixture are evenly distributed, and the base oil mixture is mixed independently until the oils in the base oil mixture are evenly distributed throughout the combined mixture. Consequentially, the essential oils mixture is mixed gradually into the base oil mixture, resulting in a combined oil mixture. The combined mixture is then applied, coating a user's scalp with a layer of oil. In embodiments, the combined oil mixture is kneaded into the scalp with squeezing and pinching motions for 1-3 minutes, and then allowed to remain on the scalp for at least 30 minutes.

In embodiments, a composition for treatment and prevention of hair loss includes a mixture of essential oils including 0.3% cedarwood essential oil, 0.3% lavender essential oil, 0.3% rosemary essential oil, 0.3% thyme essential oil, and 0.3% peppermint essential oil, and a base oil mixture of 9.85% jojoba oil, 9.85% argan oil, and 78.79% grapeseed oil.

The essential oil mixture is mixed independently until the oils in the essential oil mixture are evenly distributed, and the base oil mixture is mixed independently until the oils in the base oil mixture are evenly distributed. Subsequently, the essential oils mixture is mixed gradually into the base oil mixture.

In one embodiment, the combined oil mixture is used to coat the scalp and kneaded into the scalp with squeezing and pinching motions. The combined oil mixture is massaged into the scalp for 1-3 minutes, in embodiments, and allowed to remain on the scalp for at least 30 minutes.

In embodiments, a composition for treatment and/or prevention of hair loss includes a mixture of essential oils including 0.3% cedarwood essential oil, 0.3% lavender essential oil, 0.3% rosemary essential oil, 0.3% thyme essential oil, and 0.3% peppermint essential oil which are mixed until evenly distributed.

The composition additionally includes a base oil mixture of 9.85% jojoba oil, 9.85% argan oil, and 78.79% grapeseed oil, which base oils are mixed until evenly distributed. The essential oil mixture is subsequently mixed gradually into said base oil mixture. The combined composition is then used to coat the scalp with a thin layer, and is kneaded into the scalp with squeezing and pinching motions, for 1-3 minutes. The composition is then maintained on the scalp for at least 30 minutes.

In one embodiment of the disclosed technology, a method of treating and/or preventing hair loss includes a first step of mixing a composition of essential oils, including cedarwood essential oil, lavender essential oil, rosemary essential oil, thyme essential oil, and peppermint essential oil, jojoba oil, argan oil, and grapeseed oil until the oils are evenly distributed.

The method further includes the step of coating the scalp with the combined oil composition, and proceeding with the step of employing kneading, squeezing and pinching motions to massage the oil composition into the scalp for 1-3 minutes. The composition remains on the scalp for at least 30 minutes, in embodiments.

In another embodiment of the disclosed technology, a method includes first mixing a first composition of essential oils, including cedarwood essential oil, lavender essential oil, rosemary essential oil, thyme essential oil, and peppermint essential oil until the oils are evenly distributed. The method continues with mixing a second composition of oils, including jojoba oil, argan oil, and grapeseed oil until evenly distributed.

The first composition of essential oils is then gradually stirred into the second composition of oils, producing a combined oil composition. In an embodiment, the method proceeds with coating the scalp with said combined oil composition, and further proceeds with kneading, squeezing and pinching motions, and massaging said oil composition into the scalp for 1-3 minutes. The method, in embodiments, continues with the step of maintaining the oil composition on the scalp for at least 30 minutes.

In one embodiment, the method includes the steps of mixing a first composition of essential oils, including 0.3-5% of cedarwood essential oil, 0.3-5% of lavender essential oil, 0.3-5% rosemary essential oil, 0.3-5% thyme essential oil, 0.3-5% peppermint essential oil until the oils in the first composition are evenly distributed, and then mixing a second composition of oils, including 9.85-16.85% jojoba oil, 9.85-16.85% argan oil, and 78.79-88.79% grapeseed oil until the oils are evenly distributed.

The above method proceeds with gradually stirring the first composition of essential oils into the second composition of oils, producing a combined oil composition. In embodiments, the method further includes the step of coating the scalp with the combined oil composition. The method further proceeding, in embodiments, with the steps of kneading, squeezing and pinching the scalp coated with oil and massaging the oil composition into the scalp for 1-3 minutes. The oil composition is then allowed to remain on the scalp for at least 30 minutes.

In yet another embodiment of the disclosed technology, a method of treating and/or preventing hair loss includes the steps of mixing a first composition of essential oils, including 0.3% each of cedarwood, lavender, rosemary, thyme, peppermint essential oils until the oils are evenly distributed, and then mixing a second composition of oils, including 9.85% jojoba oil, 9.85% argan oil, and 78.79% grapeseed oil until evenly distributed. The first composition of essential oils is then gradually stirred into the second composition of oils, producing a combined oil composition.

The combined oil composition is then applied to coat the scalp with a thin layer. The above method proceeds with kneading, squeezing and pinching motions, massaging said oil composition into the scalp for 1-3 minutes. The oil composition is maintained on the scalp for at least 30 minutes. In embodiments, between 1 and 2 milliliters (mL) of the oil composition is administered at least once daily for a period of at least 3 months. After the first 3 months of daily administration, the oil composition is applied to the scalp at least 2-4 days a week.

It should be understood that the use of “and/or” is defined inclusively, such that the term “a and/or b” should be read to include the sets: “a and b,” “a or b,” “a,” “b.”

### BRIEF DESCRIPTION OF THE DRAWINGS

FIG. 1 demonstrates an image of a hair shaft and follicle with reference to the scalp.

FIG. 2 demonstrates a flow chart illustrative of steps followed to carry out a method of treating and preventing hair loss according to embodiments of the disclosed technology.

FIG. 3 includes a table listing the ingredients included in embodiments of the disclosed technology.

### DETAILED DESCRIPTION OF EMBODIMENTS OF THE DISCLOSED TECHNOLOGY

In an embodiment of the disclosed technology, a composition and method are used for treating and/or preventing hair loss naturally. A first composition of essential oils is mixed, including 0.3% each of cedarwood, lavender, rosemary, thyme, peppermint essential oils until evenly distributed. A second composition of oils is mixed, including 9.85% jojoba oil, 9.85% argan oil, and 78.79% grapeseed oil until the oils are evenly distributed. The first composition of essential oils is gradually mixed into the second composition of oils, producing a combined oil composition. The scalp is coated with said combined oil composition, proceeding with kneading, squeezing and pinching the scalp, and massaging said oil composition into the scalp for 1-3 minutes. The oil composition is then maintained on the scalp for at least 30 minutes.

The present disclosure was developed with the view that male pattern baldness is a symptom of many causes, and includes a multitude of natural ingredients to treat each individual cause of hair loss. The varied causes of hair loss can include an overproduction of dihydrotestosterone (DHT), resulting in exaggerated expression in the scalp and the excess DHT causing shrinkage or death of hair follicles. Additional causes include lowered vascular function in hair follicles which results in a lack of blood supply and lowered nutrient delivery to the hair root. The overproduction of sebum, other foreign material and infection can result in clogged hair follicles that are unable to continue in their normal growth phase.

The present disclosure was found to perform and succeed within 30 days of consistent use following the steps of the method as described therein. Additionally, embodiments of the present disclosure, when applied topically, have not caused sexual dysfunction, and neither has any of the ingredients been shown to cause sexual dysfunction, any of the side effects known to Finasteride or Minoxidil, and/or any other negative side effects.

Embodiments of the disclosed technology will become clearer in view of the following detailed description and description of the drawings.

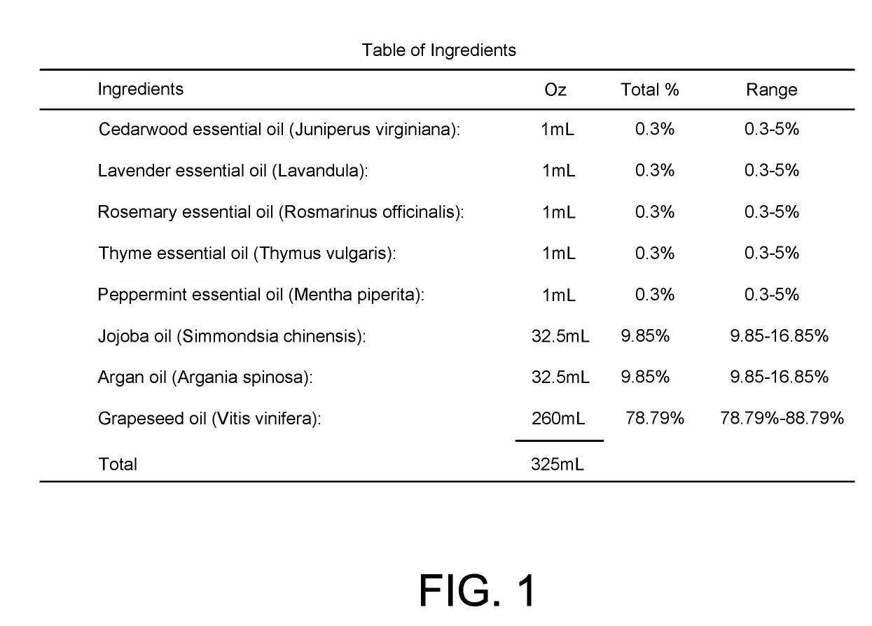

FIG. 1 shows a reference illustration with structural components of the hair follicle 9 and scalp 3. The hair shaft 1 is shown protruding from the scalp 3, the skin surface of the head. The hair follicle 9 anchors each hair shaft 1 into the skin, with the hair root 21 being the part of the hair shaft 1 below the skin. The hair follicle 9 holds the hair root 21 in a sack-like pouch. A bulb 15 forms the follicle 9 base. Blood vessels supply the hair bulb 15 with essential nutrients and hormones at the papilla 19, a loop of capillaries and connective tissue. New cells are created at the bulb 15 of the follicle 9, and the hair 1 is pushed further through a pore 13 in the scalp 3. The sebaceous gland 11 produces sebum 7 that coats the pore 13 and hair shaft 1 with an oily and wax like substance to lubricate the skin and hair 1.

The embodiments of the present invention relate to a hair loss solution that solves problems associated with hair loss and damage of the hair 1 by working from the hair root 21 and bulb 15, to the surface of the scalp 3 and the hair shaft 1. While many hair loss treatments claim to work from the root 21 to the hair shaft land scalp surface 3, many generally only address one or two alleged sources of the hair loss. Many times, a treatment may even exacerbate the source of hair loss instead of resolving the problem. Different formulations of oils claim to battle hair loss, but each tends to assume that there is only one specific cause to male pattern baldness. As such, these products address only the issue that the product assumes causes the hair loss.

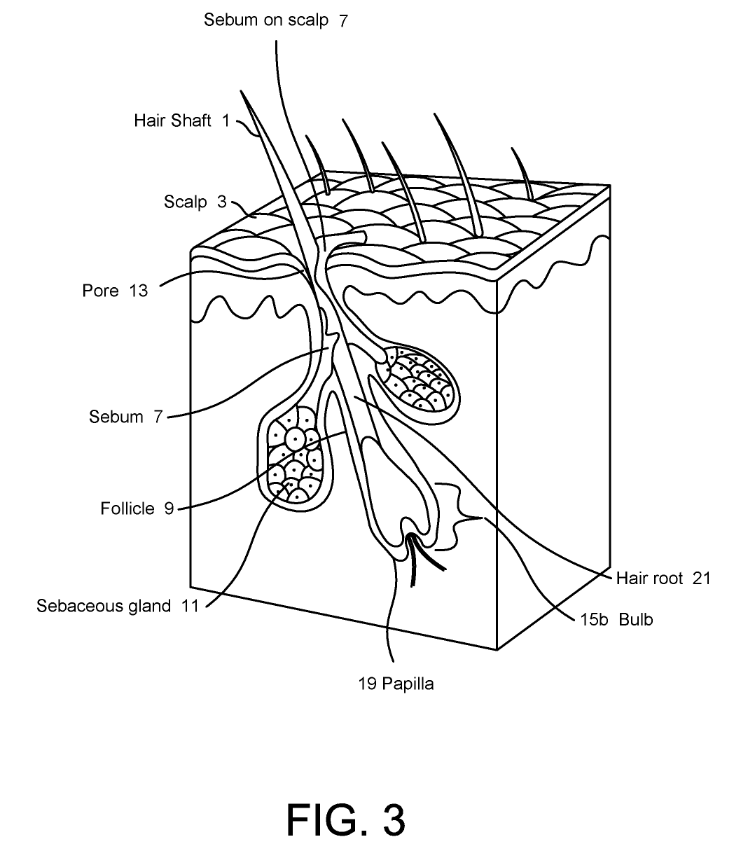

The present disclosed technology addresses each of the various roots which may be causing hair loss. Certain oils have antiseptic qualities, preventing follicles 9 from becoming clogged with foreign bodies or pus due to infection. Several oils address the overproduction of DHT, which causes hair follicles 9 to shrink and die. Oils included also treat the overproduction of sebum 7 on the scalp 3 shown in FIG. 3, since the excess sebum 7 causes clogged hair follicles 9 that are unable to continue in their normal growth phase. Particular oils of the present disclosure include nutrients essential to hair growth, and improve vascular function along the hair follicle 9 and hair root 21. Hair follicles 9 which receive the proper nutrition and blood supply allow the hair shaft 1 to continue growing and/or ensure that hair loss is minimized.

In an embodiment of the disclosed technology, a composition and method are used for treating and/or preventing hair loss naturally. The term “prevention of hair loss” as used herein refers to obstructing and inhibiting hair loss. In embodiments, the composition and method are used to specifically prevent and/or treat hair loss due to the multiple known and theorized causes of male pattern baldness. The term “hair loss” as used herein refers to the loss of hair 1 from the scalp 3 and/or the thinning of hair 1.

The present essential oil composition and method applied may prevent future hair 1 loss and contribute to the restoration of lost hair 1. The oils included in the present composition are also referred to herein as essential oils, used herein to refer to natural oils derived from the plants from which they are named, and are extracted by distillation. Each essential oil addresses multiple known and/or theorized causes of male pattern baldness. The complete natural oil composition addresses the problem of male pattern baldness from the multiple possibilities hair loss causes.

As shown in the Table of Ingredients in FIG. 1, the composition in one embodiment includes cedarwood essential oil, lavender essential oil, rosemary essential oil, thyme essential oil, peppermint essential oil, jojoba oil, argan oil, and grapeseed oil. In embodiments, the natural oil composition can include a range of ratios including between 0.3-5% cedarwood essential oil, between 0.3-5% lavender essential oil, between 0.3-5% rosemary essential oil, between 0.3-5% thyme essential oil, between 0.3-5% peppermint essential oil, between 9.85-16.85% jojoba oil, 9.85-16.85% argan oil, and 68.79-88.79% grapeseed oil.

In further embodiments, for example, the composition can include a mixture of essential oils including ratios of 0.3% cedarwood essential oil, 0.3% lavender essential oil, 0.3% rosemary essential oil, 0.3% thyme essential oil, and 0.3% peppermint essential oil, and a base oil mixture including ratios of 9.85% jojoba oil, 9.85% argan oil, and 78.79% grapeseed oil.

In embodiments, the composition is formulated with the essential oil mixture mixed independently until the essential oils are evenly distributed throughout. The term mixing as used herein is defined as combining individual components into a generally integrated whole. The term mixing, as used herein refers to, for example, but not limited to, agitating by hand, utensil, machine, shaking and/or swirling a mixture while in a container with back and forth, up and down, and/or circular motions by hand, utensil, or machine. In one embodiment, for example, holding and swirling a beaker of oils with one's hand in a circular motion will mix and evenly distribute the essential oils. The terms “evenly distributed” as used herein refer to the components of the mixture generally dispersed consistently throughout the composition.

The base oil mixture is additionally mixed independently until the oils in the base oil mixture are evenly distributed. The essential oils mixture is subsequently stirred gradually into the base oil mixture. The term “stirred” as used herein is defined as mixed and combined. The term “gradually” as used herein is defined as slowly and/or progressively. In embodiments, the oil mixture is combined when the mixture is homogenous. The term homogenous as used herein is defined as substantially uniform in color and consistency throughout.

The oil composition is used to coat the scalp 3. The term “coat” as used herein refers to covering and/or spreading a layer of said composition. In embodiments, the scalp is coated with a thin layer of the oil composition sufficient to spread over the area of interest but small enough to minimize flowing and dripping. In embodiments, the scalp is coated with an amount between 1 and 2 mL.

In an embodiment, only areas of the scalp affected by hair loss are coated with the oil composition. In other embodiments, the entire scalp 3 is coated to treat and/or prevent hair loss on all areas of the entire scalp 3. In yet further embodiments, the top of the scalp 3, prone to male pattern baldness can be coated and massaged with the oil. The oil composition is subsequently kneaded into the scalp 3 with squeezing and pinching motions. The scalp 3 is massaged with the oil for 1-3 minutes. The terms “kneaded” and “massaged” as used herein are defined as pressed, squeezed, pinched, manipulated, and/or rubbed. The terms “pinching” and “squeezing” as used herein are defined as compressing by pressing opposite sides of something such as a section of skin tissue. The oil composition is then maintained on the scalp 3 for at least 30 minutes.

A listing disclosing detailed information regarding each of the natural oils in the embodied formulations include:

Grapeseed oil (vitis vinifera) is a light oil, allowing it to be an effective base oil for mixing with other active ingredients. Grapeseed oil is additionally known to benefit healthy hair 1, as the oil contains antioxidants that have been theorized to block the production of DHT. Grapeseed oil contains vitamin E, which promotes skin and hair growth, and contains linoleic acid, a fatty acid that promotes hair growth. Grapeseed oil also contains omega-6 fatty acids, essential to hair growth. By applying these fatty acids directly to the scalp 3, prompt biological delivery is ensured, and hair growth encouraged.

Jojoba oil (simmondsia chinensis) has a chemical structure similar to that of sebum 7. Once jojoba oil is applied to the scalp 3, the similar chemical structure diminishes the scalp's 3 production of sebum 7 because the body seems to have produced enough. The application of jojoba oil to the scalp 3 has been shown to significantly cause a reduction in the sebum 7 production and oiliness of the scalp 3. This causes the hair follicles 9 that have been blocked by the overproduction of sebum 7 to begin unclogging aided also by the massage application of the natural oil composition disclosed herein. The unclogging of the hair follicles 9 allow them to return to a natural hair growth cycle. Additionally, the anti-fungal and antibacterial properties kill any bacterial or fungal sources of infection and inflammation.

Argan oil (argania spinosa) contributes to hair health, by treating and conditioning dry hair 1 and skin with fatty acids and vitamin E, and treating and preventing split ends. The anti-inflammatory properties of argan oil treat and heal inflamed and damaged follicles 9. Additional antioxidant properties of argan oil can heal damaged cells, and prevent and reverse aging symptoms.

Rosemary oil (rosmarinus officinalis) has been shown to contain compounds that lower 5-alpha-DHT levels, at a level equal to that of minoxidil, a common small molecule ingredient used in hair loss products (see Background of this patent application). Reducing DHT levels on the scalp 3 helps the scalp 3 return the hair 1 pushed into the early dormant phase back into a regular growth cycle, and prevent further hair 1 loss. Rosemary oil is also beneficial in strengthening, conditioning and healing the scalp 3 and specifically for treatment of flaky scalp, eczema, dermatitis and oily scalp.

Cedarwood oil (juniperus virginiana) promotes healthy skin and hair 1 growth. Its powerful antiseptic, anti-seborrheic, astringent and fungicidal properties making the oil an effective cure and prevention agent for infections of the scalp 3 and irritating dandruff conditions of various sources. Anti sebborrheic properties additionally halt excessive sebum 7 secretion, rectifying imbalanced functioning of the sebaceous glands. The astringent properties allow the oil to deep cleanse the scalp 3 and hair shaft 1. Consequently, the roots 21 and follicles 9 are provided with adequate air circulation.

Peppermint oil (menthe piperita) stimulates the return of blood flow to hair follicles 9. A decrease in blood supply can be a cause for hair loss, as the hair follicle 9 is deprived of the nutrients and hormones which stimulate cell division and hair growth. Increased blood flow contributes to distribution of key nutrients and hormones, stimulating cell division and hair growth.

Thyme oil (thymus vulgaris) is also known to increase blood circulation and help stimulate hair growth. Thyme oil has antiseptic properties, cleansing, renewing follicles 9 and freeing the scalp 3 of unwanted bacteria that can block up pores 13. Thyme oil contains pro vitamin A, Vitamin C, and keratin, all improving hair health and strength.

Lavender oil (Lavandula) is known to work as an antimicrobial, keeping hair follicles 9 free of bacteria. As a natural anti inflammatory, lavender oil sustains a healthy scalp 3 and can be used to remedy dandruff and psoriasis of the scalp 3. The scalp 3 is revitalized by the oil's ability to additionally enhance the scalp's 3 blood circulation in addition to treating these conditions.

The above oils, effectively, intensely, and powerfully treat hair loss by addressing the multiple causes of hair loss, as discussed. Decreased blood flow to the scalp 3 is treated with lavender oil, thyme oil, grapeseed oil and peppermint oil. Grapeseed oil, cedarwood oil, jajoba oil, and rosemary oil all reduce sebum 7 production, treating the problem of clogged hair follicles 9 due to excess production of sebum 7. Lavender, thyme, argan, cedarwood, grapeseed, and jajoba oils, known for their antiseptic qualities, also treat and cleanse the inflamed and infected follicles 9 and skin surface resulting from clogged follicles 9 due to excess sebum 7. Rosemary and grapeseed oil and thyme amongst others fight off the increased presence of dihydrotestosterone (DHT) in the scalp 3 area, which cause the shrinkage and miniaturization of hair follicles 9. The oils are additionally nutrient and vitamin rich, having nutrients necessary for cell division and healthy hair production, and addressing hair loss caused by lack of proper nutrition crucial to hair growth.

### Embodiments of a Method of Natural Oil Hair Loss Prevention and/or Treatment

In embodiments of the present disclosure, the method of natural hair loss prevention and/or treatment proceeds as described above in said hair loss system, using the composition of essential oils described above in detail. In other embodiments, this “Method of Hair Loss Prevention and/or Treatment” is not limited by the detailed description of the Hair Loss Prevention and/or Treatment oil composition and system described.

In one embodiment of the present disclosure, the method proceeds with the step of mixing a composition of essential oils, including cedarwood essential oil, lavender essential oil, rosemary essential oil, thyme essential oil, and peppermint essential oil, jojoba oil, argan oil, and grapeseed oil, until the oils are evenly distributed. Additional embodiments first include the step of mixing a specific range of ratios of the enumerated oils shown in the Table FIG. 1, a first composition including between 0.3-5% of cedarwood essential oil, between 0.3-5% of lavender essential oil, between 0.3-5% rosemary essential oil, between 0.3-5% thyme essential oil, and between 0.3-5% peppermint essential oil, and a second composition of oils including 9.85-16.85% jojoba oil, 9.85-16.85% argan oil, and 78.79-88.79% grapeseed oil.

The first and second compositions are each independently mixed, in embodiments, until the oils of the first and second compositions are evenly distributed, as defined and in embodiments, as described in the above system. The method can then proceed by stirring the first composition of essential oils gradually into the second composition of oils, producing a combined oil composition.

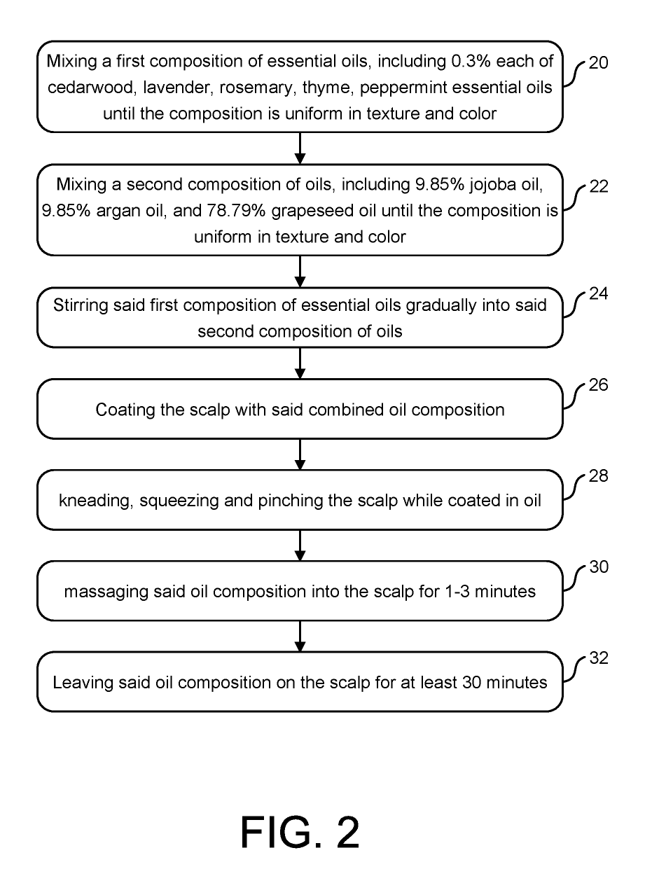

In yet further embodiments shown in the table in FIG. 1 and illustrated by FIG. 2, the method commences with the step 20 of firstly mixing specific ratios of a first composition of essential oils, including 0.3% each of cedarwood, lavender, rosemary, thyme, and peppermint essential oils until the oils are evenly distributed. Step 22 proceeds with mixing a second composition of oils, including 9.85% jojoba oil, 9.85% argan oil, and 78.79% grapeseed oil until the oils in the second composition are evenly distributed. In embodiments, the method additionally proceeds with the step 24 of stirring the first composition of essential oils gradually into the second composition of oils, producing a combined oil composition, in embodiments, as described above in the system of Natural Hair Loss Prevention and/or Treatment.

In embodiments, steps 20 through 24 proceed as described in detail above, with reference to the corresponding Hair Loss Prevention and/or Treatment System. The oil composition may be swirled and/or shaken gently for example, but not limited to, moving the container of oil in a circular motion before each use to maintain the composition's consistency.

FIG. 2 proceeds with step 26, coating the scalp 3 with the combined oil composition. In embodiments, the scalp is coated with a thin layer of the oil composition sufficient to spread over the area of interest but small enough to minimize flowing and dripping. In embodiments, the scalp is coated with an amount between 1 and 2 mL.

In an embodiment, only areas of the scalp affected by hair loss are coated with the oil composition. In other embodiments, the entire scalp 3 is coated to treat and/or prevent hair loss on all areas of the entire scalp 3. In yet further embodiments, the top of the scalp 3, prone to male pattern baldness can be coated and massaged with the oil. The oil composition is subsequently kneaded into the scalp 3 with squeezing and pinching motions. The scalp 3 is massaged with the oil for 1-3 minutes.

Step 28 proceeds with using kneading, squeezing and pinching motions to massage said oil composition into the scalp. In embodiments, the method further proceeds with step 30, massaging said oil composition into the scalp 3 for 1-3 minutes. The method additionally includes the step 32 of maintaining said oil composition on the scalp 3 for at least 30 minutes.

In embodiments, the oil composition may remain on the scalp for up to four hours. Maintaining the oil for longer than 4 hours may result in irritation of the scalp. In embodiments, the oil composition is administered at least daily for a period of at least 3 months, and subsequently, at least 2-4 days a week.

While the disclosed technology has been taught with specific reference to the above embodiments, a person having ordinary skill in the art will recognize that changes can be made in form and detail without departing from the spirit and the scope of the disclosed technology. The described embodiments are to be considered in all respects only as illustrative and not restrictive. All changes that come within the meaning and range of equivalency of the claims are to be embraced within their scope. Combinations of any of the methods and apparatuses described hereinabove are also contemplated and within the scope of the invention.

What is claimed is:

1) A composition for treatment and/or prevention of hair loss comprised of:
cedarwood essential oil, lavender essential oil, rosemary essential oil, thyme essential oil, peppermint essential oil, jojoba oil, argan oil, and grapeseed oil.

2) A composition for treatment and/or prevention of hair loss comprised of:
a mixture of essential oils including 0.3-5% cedarwood essential oil, 0.3-5% lavender essential oil, 0.3-5% rosemary essential oil, 0.3-5% thyme essential oil, and 0.3-5% peppermint essential oil, and
a base oil mixture of 9.85-16.85% jojoba oil, 9.85-16.85% argan oil, and 68.79-88.79% grapeseed oil.

3) The composition of claim 2, wherein said essential oil mixture is mixed independently until said oils of said mixture are evenly distributed and said base oil mixture is mixed independently until said oils of said base oil mixture are evenly distributed, and
wherein said essential oils mixture is further mixed gradually into said base oil mixture.

4) The composition of claim 2, wherein the composition is used to coat a user's scalp, and said oil composition is kneaded into said scalp with squeezing and pinching motions, for 1-3 minutes, and allowed to remain on said scalp for at least 30 minutes.

5) A composition for treatment and/or prevention of hair loss comprised of:
a mixture of essential oils including 0.3% cedarwood essential oil, 0.3% lavender essential oil, 0.3% rosemary essential oil, 0.3% thyme essential oil, and 0.3% peppermint essential oil, and a base oil mixture of 9.85% jojoba oil, 9.85% argan oil, and 78.79% grapeseed oil.

6) The composition of claim 5, wherein said essential oil mixture is mixed independently until evenly distributed, and said base oil mixture is mixed independently until evenly distributed, and
wherein said essential oils mixture is mixed gradually into the base oil mixture.

7) The composition of claim 5, wherein the composition of matter is used to coat the scalp with the oil composition, and the oil composition is kneaded into scalp with squeezing and/or pinching motions, for 1-3 minutes, and allowed to remain on the scalp for at least 30 minutes.

8) A composition for the treatment and/or prevention of hair loss comprised of a mixture of essential oils including 0.3% cedarwood essential oil, 0.3% lavender essential oil, 0.3% rosemary essential oil, 0.3% thyme essential oil, and 0.3% peppermint essential oil, said essential oils mixed until evenly distributed, and
a base oil mixture of 9.85% jojoba oil, 9.85% argan oil, and 78.79% grapeseed oil,
wherein said base oil mixture is mixed until evenly distributed, and
wherein said essential oil mixture is mixed gradually into said base oil mixture.

9) The composition of claim 8, wherein the composition is used to coat the scalp and is kneaded into the scalp with squeezing and/or pinching motions, for 1-3 minutes and then allowed to remain on the scalp for at least 30 minutes.

10) A composition for treatment and/or prevention of hair loss comprised of a mixture of essential oils including 0.3% cedarwood essential oil, 0.3% lavender essential oil, 0.3% rosemary essential oil, 0.3% thyme essential oil, and 0.3% peppermint essential oil, said essential oils mixed until evenly distributed, and
a base oil mixture of 9.85% jojoba oil, 9.85% argan oil, and 78.79% grapeseed oil, said base oils mixed until evenly distributed,
wherein said essential oil mixture is mixed gradually into said base oil mixture, and
wherein said composition is used to coat the scalp and is kneaded into the scalp with squeezing and pinching motions, for 1-3 minutes and then allowed to remain on the scalp for at least 30 minutes.

11) A method of treating and/or preventing hair loss, comprising the steps of:
mixing a composition of essential oils, including cedarwood essential oil, lavender essential oil, rosemary essential oil, thyme essential oil, and peppermint essential oil, jojoba oil, argan oil, and grapeseed oil until evenly distributed.

12) The method of claim 11, further comprising the steps of coating the scalp with said combined oil composition, proceeding with kneading, squeezing and pinching motions, massaging said oil composition into the scalp for 1-3 minutes, and maintaining said composition on the scalp for at least 30 minutes.

13) A method of treating and/or preventing hair loss, comprising the steps of:
mixing a first composition of essential oils, including cedarwood essential oil, lavender essential oil, rosemary essential oil, thyme essential oil, and peppermint essential oil until evenly distributed,
mixing a second composition of oils, including jojoba oil, argan oil, and grapeseed oil until evenly distributed, and
stirring said first composition of essential oils gradually into said second composition of oils, producing a combined oil composition.

14) The method of claim 13, further comprising the steps of coating the scalp with said combined oil composition, and proceeding with kneading, squeezing and pinching motions, massaging said oil composition into the scalp for 1-3 minutes.

15) The method of claim 14, additionally comprising the step of maintaining said oil composition on the scalp for at least 30 minutes.

16) A method of treating and/or preventing hair loss, the method comprising the steps of:
mixing a first composition of essential oils, including 0.3-5% of cedarwood essential oil, 0.3-5% of lavender essential oil, 0.3-5% rosemary essential oil, 0.3-5% thyme essential oil, 0.3-5% peppermint essential oil until evenly distributed,
mixing a second composition of oils, including 9.85-16.85% jojoba oil, 9.85-16.85% argan oil, and 78.79-88.79% grapeseed oil until evenly distributed, and
stirring said first composition of essential oils gradually into said second composition of oils, producing a combined oil composition.

17) The method of claim 16, further comprising the steps of coating the scalp with said combined oil composition.

18) The method of claim 17, further proceeding with the steps of kneading, squeezing and pinching motions, massaging said oil composition into the scalp for 1-3 minutes, and maintaining said oil composition on the scalp for at least 30 minutes.

19) A method of treating and/or preventing hair loss, the method comprising the steps of:
mixing a first composition of essential oils, including 0.3% each of cedarwood, lavender, rosemary, thyme, peppermint essential oils until evenly distributed,
mixing a second composition of oils, including 9.85% jojoba oil, 9.85% argan oil, and 78.79% grapeseed oil until evenly distributed,
stirring said first composition of essential oils gradually into said second composition of oils, producing a combined oil composition,
coating the scalp with said combined oil composition,
proceeding with kneading, squeezing and pinching motions, massaging said oil composition into the scalp for 1-3 minutes, and
maintaining said oil composition on the scalp for at least 30 minutes.

20) The method of claim 19 wherein between 1 and 2 milliliters (mL) of said oil composition is administered at least daily for a period of at least 3 months, and subsequently, at least 2-4 days a week.
